(19) United States
(12) Patent Application Publication
(10) Pub. No.: US 2021/0062217 A1
(43) Pub. Date: Mar. 4, 2021

(54) BIDIRECTIONAL CHEF1 VECTORS

(71) Applicant: AGC BIOLOGICS, INC., Bothell, WA (US)

(72) Inventor: Sandhya Pande, Bothell, WA (US)

(21) Appl. No.: 16/960,370

(22) PCT Filed: Jan. 9, 2019
(86) PCT No.: PCT/US19/12833

§ 371 Date: Jul. 7, 2020

Related U.S. Application Data
(60) Provisional application No. 62/615,574, filed on Jan. 10, 2018.

(51) Int. Cl. C12N 15/85

(52) U.S. Cl. CPC C12N 15/85; C12N 2830/85; C12N 2830/205

(57) ABSTRACT

The invention provides bidirectional expression vectors comprising Chinese hamster ovary elongation factor 1-a (CHEF1) transcriptional regulatory DNA elements, a gene of interest (GO I), a minimal cytomegalovirus (minCMV) and a selectable marker (SM) and/or a human adenovirus tripartite leader (AdTPL) sequence. The invention also provides method for increasing heterologous protein expression in a host cell comprising culturing the host cell the bidirectional expression vector(s).

This application contains, as a separate part of the disclosure, a sequence listing in computer-readable form (Filename: 52530_Seqlisting.txt; Size: 129,138 bytes; Created: Jan. 8, 2019) which is incorporated by reference in its entirety.

### FIELD OF THE INVENTION

This invention is directed to bidirectional expression vectors comprising novel promoter-enhancer combinations that increase heterologous protein expression and has practical applications in the field of recombinant protein production.

### BACKGROUND OF THE INVENTION

Increasing recombinant protein expression through improvements in transcription, translation, protein folding and/or secretion is a fundamental priority for optimizing yield during cell line development. The Chinese hamster ovary elongation factor 1-α (CHEF1) expression system has been used extensively to create clinical cell lines for producing recombinant proteins. The elongation factor 1-α (EF-1α) gene is highly expressed in most tissue types, and EF-1 is one of the most abundant proteins in human cells (Beck et al., Molecular Systems Biology 7: 549; 2011). CHEF1 expression vectors achieve high-level recombinant protein expression in Chinese hamster ovary (CHO) cells, as well as in non-hamster cells.

CHEF1 expression is coordinated with growth such that titer increases are driven by increased volumetric productivity. Typically, protein expression initiates early in the exponential phase of growth and drops off during stationary phase and decline. The linkage between protein expression and cell growth is consistent with the regulation of the native EF-1α gene, which is constitutively expressed to function in ribosomal protein complexes. Expression of EF-1α has been documented to increase in transformed (Sanders et al., Nucleic Acids Research 20: 5907; 1992) and mitogen-stimulated cells (Thomas and Thomas, Journal of Cell Biology 103: 2137; 1986), consistent with constitutive expression of EF-1α in actively growing cells. In addition to transcriptional control in growing cells, the growth factor insulin regulates the translation of EF-1α through the mRNA 5′ untranslated region (5′UTR) (Hammond and Bowman, Journal of Biological Chemistry 25: 17785; 1988; Proud and Denton, Biochemical Journal 328: 329; 1997). This translational control is achieved through the Tract of Polypyrimidine (TOP) sequence found in the 5′UTR (Mariottini and Amaldi, Molecular and Cellular Biology 10: 816; 1990).

CHEF1 expression systems have shown to be capable of achieving higher levels of protein expression than vectors employing other commonly used promoters, such as the cytomegalovirus (CMV), human EF-1α, and Simian virus 40 (SV40) promoters (Running Deer and Allison, Biotechnology Progress 20: 880; 2004). The CMV promoter is one of the most widely used promoters for recombinant protein expression. For example, the glutamine synthetase (GS) system uses a murine or human CMV promoter (Kalwy, S., “Towards stronger gene expression—a promoter's tale,” 19^{th} European Society for Animal Cell Technology (ESACT) meeting, 2005). The commercial expression plasmid pcDNA™3 (Life Technologies Corp., Carlsbad, Calif.) carries a CMV promoter derived from the major immediate-early (IE) gene (GenBank Accession #K03104.1) described previously (Boshart et al., Cell 1985; 4:521). Another commonly used CMV promoter is derived from the human CMV strain AD169 (GenBank Accession #X17403.1), also known as human herpesvirus 5.

Vectors containing CHEF1 regulatory DNA result in improved expression of recombinant proteins that is up to 280-fold greater than from CMV-controlled plasmids (Running Deer and Allison, 2004). Increased expression of a variety of proteins of interest, including secreted and membrane-bound proteins, has been achieved in different eukaryotic cell lines, including non-hamster cells, using CHEF1-driven vectors. Transfection efficiencies between CHEF1 and CMV vectors are comparable, but expression levels in clones transfected with CHEF1 vectors are generally uniformly higher.

Despite the demonstrated success of CHEF1 vectors in driving high-level expression of recombinant proteins, there exists an ongoing need to develop improved expression systems.

### SUMMARY OF THE INVENTION

The disclosure provides bidirectional expression vectors for high-level expression of one or more recombinant proteins and/or a selectable marker (SM). In various aspects, the bidirectional expression vector comprises CHEF1 transcriptional regulatory DNA elements, a gene of interest (GOI), and a selectable marker (SM). In some aspects, the bidirectional expression vectors further comprise a CMV promoter and/or a human adenovirus tripartite leader (AdTPL) sequence. In a related aspect, the bidirectional expression vector comprises a minimal cytomegalovirus promoter (minCMV).

In various embodiments, the disclosure provides a method for increasing heterologous protein expression in a host cell comprising the steps of culturing the host cell comprising the bidirectional expression vector.

In various aspects, a bidirectional expression vector according to the disclosure comprises CHEF1 transcriptional regulatory DNA and a GOI. In various embodiments, the orientation of the CHEF1 transcriptional regulatory DNA and the GOI are 5′: 3′ (i.e. the CHEF1 transcriptional regulatory DNA and the GOI are in the same orientation). In various aspects, the 5′ CHEF1 transcriptional regulatory DNA comprises Sequence ID NO: 3 or a polynucleotide at least 95% identical to Sequence ID NO: 3. In various embodiments the 5′ CHEF1 transcriptional regulatory DNA comprises Sequence ID NO: 4 or a polynucleotide at least 95% identical to Sequence ID NO: 4.

In various aspects, a bidirectional expression vector according to the disclosure further comprises 3′ CHEF1 transcriptional regulatory DNA wherein the 3′ CHEF1 transcriptional regulatory DNA is in the same orientation as the 5′ CHEF1 transcriptional regulatory DNA. In various embodiments, the 3′ CHEF1 transcriptional regulatory DNA comprises SEQ ID NO: 5 or a polynucleotide at least 95% identical to Sequence ID NO: 5.

In various aspects, a bidirectional expression vector according to the disclosure comprises a minimal CMV (minCMV) and the selectable marker (SM). In various embodiments, the orientation of the minCMV and the SM are 3′: 5′(i.e. the CHEF1 transcriptional regulatory DNA and the GOI are in reverse orientation relative to the minCMV and SM). In various embodiments, the SM is codon deoptimized.

In various aspects, a bidirectional expression vector according to the disclosure comprises a CHEF1 transcriptional regulatory DNA, a GOI, and a SM. In related aspects, the orientation of the CHEF1 transcriptional regulatory DNA and the GOI are 5′: 3′ (i.e. the CHEF1 transcriptional regulatory DNA and the GOI are in same orientation). In various embodiments, the 5′ CHEF1 transcriptional regulatory DNA comprises Sequence ID NO: 3 or a polynucleotide at least 95% identical to Sequence ID NO: 3. In related embodiments, the bidirectional expression vector further comprises 3′ CHEF1 transcriptional regulatory DNA wherein the 3′ CHEF1 transcriptional regulatory DNA is in the same orientation as the 5′ CHEF1 transcriptional regulatory DNA and the GOI. In related embodiments, the 3′ CHEF1 transcriptional regulatory DNA comprises Sequence ID NO: 3 or a polynucleotide at least 95% identical to Sequence ID NO: 3. In related embodiments, the orientation of the SM is 3′: 5′ (i.e., in reverse orientation relative to the 5′ CHEF1 transcriptional regulatory DNA, 3′ CHEF1 transcriptional regulatory DNA and GOI. In related embodiments, the SM is upstream of the CHEF1 transcriptional regulatory DNA. In related embodiments, the SM is codon deoptimized.

In various aspects, a bidirectional expression vector according to the disclosure comprises CHEF1 transcriptional regulatory DNA and a CMV promoter and/or a human adenovirus tripartite leader (AdTPL) sequence, a GOI, a minCMV and a SM.

In various aspects, a bidirectional expression vector according to the disclosure comprises CHEF1 transcriptional regulatory DNA and a CMV promoter, a GOI and a SM.

In various embodiments, a bidirectional expression vector according to the disclosure further comprises a selectable marker gene. In various aspects, the SM is codon deoptimized. In various aspects, the SM is codon deoptimized. In various aspects, the SM is selected from the group consisting of neomycin phosphotransferase (npt II), hygromycin phosphotransferase (hpt), dihydrofoate reductase (dhfr), zeocin, phleomycin, bleomycin resistance gene ble (enzyme not known), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2) and orotidine-5′-phosphate (OMP) decarboxylase (ura3).

In various embodiments, the disclosure provides methods for increasing heterologous protein expression in a host cell comprising the steps of culturing the host cell comprising a bidirectional expression vector according to the disclosure. In various aspects, the host cell is a eukaryotic or prokaryotic cell (e.g. Escherichia coli). In various aspects, the host cell is a yeast cell (e.g. Saccharomyces cerevisiae or Pichia pastoris). In various aspects, the host cell is an insect cell (e.g Spodoptera frugiperda). In various aspects, the host cell is a plant cell. In various aspects, the host cell is a protozoan cell. In various aspects, the host cell is a In various aspects, the host cell is a mammalian cell. In various aspects, the host cell is a human cell. In various aspects, the host cell is a Chinese hamster cell. In various aspects, the host cell is a Chinese hamster ovary cell (CHO). In various aspects, the host cell is a serum-free, suspension-adapted CHO cell line (SFSA DG44).

### BRIEF DESCRIPTION OF THE DRAWINGS

FIGS. 1A-1C show maps of the bidirectional expression vector pDEF90 (FIG. 1A), pDEF90 minus CMV (FIG. 1B), and pDEF90 minus CHEF (FIG. 1C). FIG. 1A shows pDEF90 comprising 5′ and 3′ CHEF1 transcriptional regulatory DNA and a minimal CMV (minCMV) promoter. The minCMV promoter is present in the reverse orientation upstream of the CHEF promoter. The DHFR gene is present downstream of minimal CMV element in the reverse orientation with respect to CHEF promoter. FIG. 1B shows pDEF90 minus CMV comprising 5′ and 3′ CHEF1 transcriptional regulatory DNA. The minCMV promoter has been removed from the pDEF90 vector. The DHFR gene is present in the reverse orientation upstream of CHEF promoter. FIG. 1C shows pDEF90 minus CHEF, in which 4 kb of the 5′ CHEF1 transcriptional regulatory DNA has been removed. pDEF90 comprises 3′ CHEF1 transcriptional regulatory DNA and the DHFR gene is present downstream of minimal CMV promoter.

FIGS. 2A-2D show transfection of pDEF90 vectors and their effects on cell growth, viability, doubling time and generations of transfection pools during recovery and selection over 21 days. The viable cell density (FIG. 2A), viability (FIG. 2B), cell doubling time (FIG. 2C), number of generations (FIG. 2D) for pDEF38; pDEF90; pDEF90 minus CHEF; pDEF90 minus CMV and a mock control.

FIGS. 3A-3C show that pDEF90 drives expression of heterologous protein in Serum Free, Suspension Adapted (SFSA) cells. The viable cell density (FIG. 3A) and viability (FIG. 3B) are shown following 0-21 days post transfection of SFSA cells with pDEF90-GFP-1 and pDEF90-GFP-2 (duplicate transfections in SFSA cells with linearized plasmid). Transfectants were recovered in CDCIM1 media and GFP was measured using Guava flowcytometer. FIG. 3C shows the count of two pDEF90-GFP transfection pools, with measurement of Green fluorescence (GRN-HLog). pDEF90-GFP pool and pDEF90 (without GFP) transfected pool.

FIG. 4 shows GFP expression of pEF90-GFP clones. 12 clones were analyzed for GFP expression by flow cytometry. Left peak: untransfected cells; Right peak: pDEF90-GFP clones.

### DETAILED DESCRIPTION OF THE INVENTION

The present disclosure provides various bidirectional expression vectors comprising, in various aspects, Chinese Hamster Elongation Factor-1a (CHEF1) transcriptional regulatory DNA, a gene of interest (GOI) and a selectable marker (SM). In related aspects, the bidirectional expression vectors may also comprise a minimal cytomegalovirus (minCMV) promoter, a cytomegalovirus (CMV) promoter and/or a human adenovirus tripartite leader (AdTPL) sequence.

The use of CHEF1 transcriptional regulatory DNA elements in an expression vector (unidirectional expression vectors) to achieve high-level expression of recombinant proteins has been previously described (U.S. Pat. Nos. 5,888,809; 9,212,367; 9,297,024 (each of which are hereby incorporated by reference in their entirety); Running Deer and Allison, 2004). Protein expression from CHEF1-driven vectors has been shown to be significantly higher than from CMV promoter-controlled vectors for a number of different protein and host cell types, and the increase can be greater than 250-fold (Running Deer and Allison, 2004). The AdTPL sequence is a 200-nucleotide 5′ noncoding sequence found on late viral mRNAs that enhances their translation (Logan, PNAS 81: 3655; 1984).

### Definitions

As used herein, the following definitions may be useful in aiding the skilled practitioner in understanding the disclosure:

The term “bidirectional,” as used herein, refers to the expression of a gene of interest or selectable marker in both 5′ to 3′ (transcription direction) and 3′ to 5′ (respective opposite transcription direction). The term “bidirectional expression vector” refers to an expression vector in which the expression cassettes are organized such that the first expression cassette and the second expression cassette are arranged in opposite direction, i.e. the expression cassette for a gene of interest (GOI) in one transcription direction and the expression cassette for a selectable marker (SM) is in the respective opposite transcription direction.

The term “expression vector” or “vector” refers to any molecule used to transfer coding information to a host cell. In various aspects, the expression vector is a nucleic acid, a plasmid, a cosmid, a virus, or an artificial chromosome. An “expression plasmid” or “plasmid” according to the disclosure is further described in the Examples.

The term “deoptimized” as used herein with reference to a polynucleotide means that the polynucleotide has been modified in such a way that translation of a protein encoded by the polyncleotide is less than optimal for the host cell in which the polyncleotide has been introduced. A polynucleotide is deoptimized in a multitude of ways and the present invention is not limited by the methods exemplified herein.

The term “host cell” refers to a cell that has been transformed, transfected, or transduced by a bidirectional expression vector bearing a GOI, which is then expressed by the cell. A host cell is, in various aspects, a prokaryotic or eukaryotic cell. In various aspects, the host cell is a bacteria cell, a protist cell, a fungal cell, a plant cell, or an animal cell. The term also refers to progeny of the parent host cell, regardless of whether the progeny is identical in genotype or phenotype to the parent, as long as the gene of interest is present.

The terms “cytomegalovirus promoter” or “CMV promoter” refer to CMV promoter sequences known in the art. In various aspects, the CMV promoter is of any origin, including murine (mCMV) or human (hCMV). In various aspects, a hCMV is derived from any CMV strain. In various aspects, the CMV strain is AD169, Davis, Toledo, or Towne. In various embodiments of the disclosure, the CMV promoter contains the polynucleotide set forth in SEQ ID NO: 1.

The terms “minimal CMV” or “minCMV” promoters, refer to, the minimal elements of a CMV promoter, including the TATA box and transcription initiation site, which is inactive (or has very low basal activity) unless regulatory elements that enhance promoter activity are placed upstream. An example of a minCMV promoter for use in the instant disclosure, includes the polynucleotide set forth in SEQ ID NO: 6.

The term “AdTPL sequence” refers to the approximately 200 nucleotide, 5′ noncoding sequence present in human adenovirus late viral mRNAs that is known in the art. In various embodiments, the AdTPL sequence contains the polynucleotide set forth in SEQ ID NO: 2.

The term “transcriptional regulatory DNA” refers to noncoding sequences containing cis-acting regulatory elements capable of controlling transcription of a gene, such as the promoter region and elements such as enhancers, insulators, and scaffold/matrix attachment regions.

The term “CHEF1 transcriptional regulatory DNA” refers to noncoding sequences containing cis-acting regulatory elements capable of controlling transcription of the CHEF1 gene, such as the promoter region and elements such as enhancers, insulators, and scaffold/matrix attachment regions.

The term “5′ CHEF1 transcriptional regulatory DNA” refers to DNA, when in nature, located 5′, i.e., upstream, of the start codon in the CHEF1 gene in the Chinese hamster genome.

The term “3′ CHEF1 transcriptional regulatory DNA” refers to DNA, when in nature, located 3′, i.e., downstream, of the stop codon in the CHEF1 gene in the Chinese hamster genome.

The terms “approximately” and “about” refer to quantities that are within close range of a reference amount. With respect to polynucleotides, a sequence that is approximately/about a specified length is within 5% of the recited length.

### Bidirectional Expression Vectors

Bidirectional expression vectors are designed to constitutively express one or more genes of interest and optionally a selectable marker. In various aspects, bidirectional vectors may encode one or more promoters. In various aspects, the expression vectors comprise one, two, three or four genes of interest. In related aspects, the one, two, three, or four genes of interest are under the control of one or optionally two promoters.

The bidirectional expression vectors of the invention allow for enhanced stability of a gene of interest (GOI) as the selection marker (DHFR) and the GOI are expressed from the same CHEF1 promoter.

pDEF90

In various aspects, a bidirectional expression vector according to the disclosure comprises a CHEF1 transcriptional regulatory DNA, a minCMV, a GOI, and a SM.

In various aspects, a bidirectional expression vector according to the disclosure comprises CHEF1 transcriptional regulatory DNA and the GOI in 5′: 3′ orientation (i.e. the CHEF1 transcriptional regulatory DNA and the GOI are in same orientation). In various embodiments, the 5′ CHEF1 transcriptional regulatory DNA comprises Sequence ID NO: 3. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 3. In various embodiments, the 5′ CHEF1 transcriptional regulatory DNA comprises the polynucleotide set forth in SEQ ID NO: 4. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 4.

In various aspects, a bidirectional expression vector according to the disclosure comprises the minCMV and the SM in 3′: 5′ orientation (i.e. the minCMV and the SM are in reverse orientation relative to the CHEF1 transcriptional regulatory DNA and the GOI). In various aspects, the SM is codon deoptimized. In various embodiments of the disclosure, the minCMV promoter contains the polynucleotide set forth in SEQ ID NO: 6.

In various aspects, a bidirectional expression vector according to the disclosure further comprises a 3′ CHEF1 transcriptional regulatory DNA. In various embodiments, the 3′ CHEF1 transcriptional regulatory DNA comprises Sequence ID NO: 5. The disclosure also provides 3′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 5.

pDEF90 Minus CMV

In various aspects, a bidirectional expression vector according to the disclosure comprises a CHEF1 transcriptional regulatory DNA, a GOI, and a SM. In various aspects, a bidirectional expression vector according to the disclosure comprises CHEF1 transcriptional regulatory DNA and the GOI in 5′: 3′ orientation (i.e. the CHEF1 transcriptional regulatory DNA and the GOI are in the same orientation). In related embodiments, the 5′ CHEF1 transcriptional regulatory DNA comprises Sequence ID NO: 3. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 3. In various embodiments, the 5′ CHEF1 transcriptional regulatory DNA comprises the polynucleotide set forth in SEQ ID NO: 4. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 4.

In related aspects, a bidirectional expression vector according to the disclosure further comprises a 3′ CHEF1 transcriptional regulatory DNA. In various embodiments, the 3′ CHEF1 transcriptional regulatory DNA comprises Sequence ID NO: 5 The disclosure also provides 3′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 5.

In related aspects, a bidirectional expression vector according to the disclosure comprises the SM in 3′: 5′ orientation (i.e. the SM is in reverse orientation relative to the 5′ CHEF1 transcriptional regulatory DNA, 3′ CHEF1 transcriptional regulatory DNA, and the GOI). In various aspects, SM is upstream of the CHEF1 transcriptional regulatory DNA. In various aspects, the SM is codon deoptimized. In related aspects, the aforementioned CHEF1 transcriptional regulatory DNA sequences promote the expression of both the SM and GOI.

pDEF90 CHEF CMV AdTPL Hybrid

In various aspects, a bidirectional expression vector according to the disclosure comprises a CHEF1 transcriptional regulatory DNA and a CMV promoter and/or a human adenovirus tripartite leader (AdTPL) sequence, a GOI, a minCMV and a SM. In various aspects, the SM is codon deoptimized. In various embodiments of the disclosure, the AdTPL contains the polynucleotide set forth in SEQ ID NO: 2.

In related aspects, a bidirectional expression vector according to the disclosure further comprises a 3′ CHEF1 transcriptional regulatory DNA. In related embodiments, the 3′ CHEF1 transcriptional regulatory DNA comprises Sequence ID NO: 5. The disclosure also provides 3′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 5.

pDEF90 with CHEF and CMV

In various aspects, a bidirectional expression vector according to the disclosure comprises a CHEF1 transcriptional regulatory DNA and a CMV promoter, a GOI and a SM. In various aspects, the SM is codon deoptimized. In various aspects of the disclosure, the CMV promoter contains the polynucleotide set forth in SEQ ID NO: 1.

In various embodiments, the 5′ CHEF1 transcriptional regulatory DNA comprises SEQ ID NO: 3. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 3. In various embodiments, the 5′ CHEF1 transcriptional regulatory DNA comprises the polynucleotide set forth in SEQ ID NO: 4. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 4.

In various aspects, a bidirectional expression vector according to the disclosure further comprises a 3′ CHEF1 transcriptional regulatory DNA. In various embodiments, the 3′ CHEF1 transcriptional regulatory DNA comprises SEQ ID NO: 5. The disclosure also provides 3′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 5.

pDEF93CMV

In various aspects, a bidirectional expression vector according to the disclosure comprises a CHEF1 transcriptional regulatory DNA, a minCMV, a GOI, and a SM with a 5′ UTR and a SV40 polyadenylation sequence at the 3′ end of SM for efficient mRNA processing.

In various aspects, a bidirectional expression vector according to the disclosure comprises CHEF1 transcriptional regulatory DNA and the GOI in 5′: 3′ orientation (i.e. the CHEF1 transcriptional regulatory DNA and the GOI are in the same orientation). In various embodiments, the 5′ CHEF1 transcriptional regulatory DNA comprises Sequence ID NO: 3. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 3. In various embodiments, the 5′ CHEF1 transcriptional regulatory DNA comprises the polynucleotide set forth in SEQ ID NO: 4. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 4.

In various aspects, a bidirectional expression vector according to the disclosure comprises the minCMV and the SM in 3′: 5′ orientation (i.e. the minCMV and the SM are in reverse orientation relative to the CHEF1 transcriptional regulatory DNA and the GOI). In various aspects, the SM is codon deoptimized. In various embodiments of the disclosure, the minCMV promoter contains the polynucleotide set forth in SEQ ID NO: 6.

In various aspects, a bidirectional expression vector according to the disclosure further comprises a 3′ CHEF1 transcriptional regulatory DNA. In various embodiments, the 3′ CHEF1 transcriptional regulatory DNA comprises Sequence ID NO: 5. The disclosure also provides 3′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 5.

pDEF94CMV

In various aspects, a bidirectional expression vector according to the disclosure comprises a CHEF1 transcriptional regulatory DNA, a minCMV, a GOI, and a SM with a SV40 polyadenylation sequence at the 3′ end of SM for efficient mRNA processing.

In various aspects, a bidirectional expression vector according to the disclosure comprises CHEF1 transcriptional regulatory DNA and the GOI in 5′: 3′ orientation (i.e. the CHEF1 transcriptional regulatory DNA and the GOI are in same orientation). In various embodiments, the 5′ CHEF1 transcriptional regulatory DNA comprises Sequence ID NO: 3. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 3. In various embodiments, the 5′ CHEF1 transcriptional regulatory DNA comprises the polynucleotide set forth in SEQ ID NO: 4. The disclosure also provides 5′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 4.

In various aspects, a bidirectional expression vector according to the disclosure comprises the minCMV and the SM in 3′: 5′ orientation (i.e. the minCMV and the SM are in reverse orientation relative to the CHEF1 transcriptional regulatory DNA and the GOI). In various aspects, the SM is codon deoptimized. In various embodiments of the disclosure, the minCMV promoter contains the polynucleotide set forth in SEQ ID NO: 6.

In various aspects, a bidirectional expression vector according to the disclosure further comprises a 3′ CHEF1 transcriptional regulatory DNA. In various embodiments, the 3′ CHEF1 transcriptional regulatory DNA comprises Sequence ID NO: 5. The disclosure also provides 3′ CHEF1 transcriptional regulatory DNA that is at least 99%, at least 98%, at least 97%, at least 96%, at least 95%, at least 94%, at least 93%, at least 92%, at least 91%, at least 90%, at least 85%, at least 80%, at least 75% or at least 70% identical to the polynucleotide set out in SEQ ID NO: 5.

### Selectable Marker

Selectable markers are used in transfection experiments to complement host cell protein deficiencies or confer resistance to an otherwise toxic agent, and thereby select for the presence (expression) of co-transformed genes of interest.

In various aspects, the bidirectional expression vector further comprises a selectable marker (SM) gene for identification of transformed cells. Examples of suitable SM genes include, but are not limited to, neomycin phosphotransferase (npt II), hygromycin phosphotransferase (hpt), dihydrofolate reductase (dhfr), zeocin, phleomycin, bleomycin resistance gene (ble), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2), orotidine-5′-phosphate (OMP) decarboxylase (ura3), and orthologs of any of the foregoing.

The disclosure also provides host cells transformed, transduced, or transfected with a bidirectional expression vector comprising CHEF1 transcriptional regulatory DNA and a CMV promoter and/or an AdTPL sequence. In various aspects, the host cell is a prokaryotic or eukaryotic cell. In various aspects, the host cell is a hamster cell. In various aspects, the hamster cell is a CHO cell. In various embodiments, the host cell is a non-hamster mammalian cell, and in various aspects, the cell is a human cell.

### Codon Deoptimization

In various aspects, the SM is codon deoptimized. Methods for codon optimization have been described by others (Itakura 1987, Kotula 1991, Holler 1993, Seed 1998). However, there are limited examples of codon deoptimization utility. One such example is the deoptimization of virus genes to reduce replicative fitness by incorporating least preferred codons or nonrandomized codon pairs (Burns 2006, Mueller 2006, Coleman 2008, Kew 2008). Methods of codon deoptimization is further described in U.S. Pat. No. 9,212,367, which is incorporated by reference, with particular reference to the examples, described the codon deoptimized DHFR-encoding polynucleotide sequences were introduced into the CHEF1 expression vector pDEF38, (Columns 14-16). The examples presented are generally applicable to deoptimize codons in a polynucleotide encoding any selectable marker for its species specific host.

### Genes of Interest

In various aspects, the bidirectional expression vector further comprises one or more genes of interest (GOI). Examples of suitable GOI include, but are not limited to, monoclonal or polyclonal antibodies and other glycoproteins, biosimilars, Fc-fusion genes, enzymes, vaccines, peptide hormones, or growth factors. In related aspects, for antibodies, the heavy chain (HC) and light chain (LC) can be expressed in a single vector from a single promoter.

### Vectors and Host Cells

Any eukaryotic and prokaryotic vector is contemplated for use in the instant methods, including mammalian, yeast, fungal, insect, plant or viral vectors useful for selected host cell. The term “vector” is used as recognized in the art to refer to any molecule (e.g., nucleic acid, plasmid, or virus) used to transfer coding information to a host cell. The term “host cell” is used to refer to a cell which has been transformed, or is capable of being transformed, by a vector bearing a selected gene of interest which is then expressed by the cell. The term includes mammalian, yeast, fungal, insect, plant and protozoan cells, and the progeny of the parent cell, regardless of whether the progeny is identical in morphology or in genetic make-up to the original parent, so long as the selected gene is present. In general, any vector can be used in methods of the invention and selection of an appropriate vector is, in one aspect, based on the host cell selected for expression of the GOI.

Examples include, but are not limited to, mammalian cells, such as Chinese hamster ovary cells (CHO) (ATCC No. CCL61); CHO DHFR-cells; serum-free, suspension-adapted CHO DHFR cell line was created at CMC ICOS (SFSA DG44 cells); human embryonic kidney (HEK) 293 or 293T cells (ATCC No. CRL1573); or 3T3 cells (ATCC No. CCL92). Other suitable mammalian cell lines, are the monkey COS-1 (ATCC No. CRL1650) and COS-7 (ATCC No. CRL1651) cell lines, and the CV-1 cell line (ATCC No. CCL70). Still other suitable mammalian cell lines include, but are not limited to, Sp2/0, NS1 and NS0 mouse hybridoma cells, mouse neuroblastoma N2A cells, HeLa, mouse L-929 cells, 3T3 lines derived from Swiss, Balb-c or NIH mice, BHK or HaK hamster cell lines, which are also available from the ATCC.

Further exemplary mammalian host cells include primate cell lines and rodent cell lines, including transformed cell lines. Normal diploid cells, cell strains derived from in vitro culture of primary tissue, as well as primary explants, are also suitable.

Similarly useful as host cells include, for example, the various strains of E. coli (e.g., HB101, (ATCC No. 33694) DH5ÿ, DH10, and MC1061 (ATCC No. 53338)), various strains of B. subtilis, Pseudomonas spp., Streptomyces spp., Salmonella typhimurium and the like.

Many strains of yeast cells known to those skilled in the art are also available as host cells for expression of a GOI and include, for example, Saccharomyces cerevisiae, Schizosaccharomyces pombe, Kluyveromyces strains, Candida, Pichia ciferrii and Pichia pastoris.

Additionally, where desired, insect cell systems may be utilized in the methods of the present invention. Such systems include for example and without limitation, Sf-9 and Hi5 (Invitrogen, Carlsbad, Calif.).

Exemplary fungal cells include, without limitation, Thermoascus aurantiacus, Aspergillus(filamentous fungus), including without limitation Aspergillus oryzaem, Aspergillus nidulans, Aspergillus terreus, and Aspergillus niger, Fusarium (filamentous fungus), including without limitation Fusarium venenatum, Penicillium chrysogenum, Penicillium citrinum, Acremonium chrysogenum, Trichoderma reesei, Mortierella alpina, and Chrysosporium lucknowense.

Exemplary protozoan cells include without limitation Tetrahymena strains and Trypanosoma strains.

An expression plasmid according to the disclosure is further described in the following Example. The Example serves only to illustrate the invention and is not intended to limit the scope of the invention in any way.

### EXAMPLES

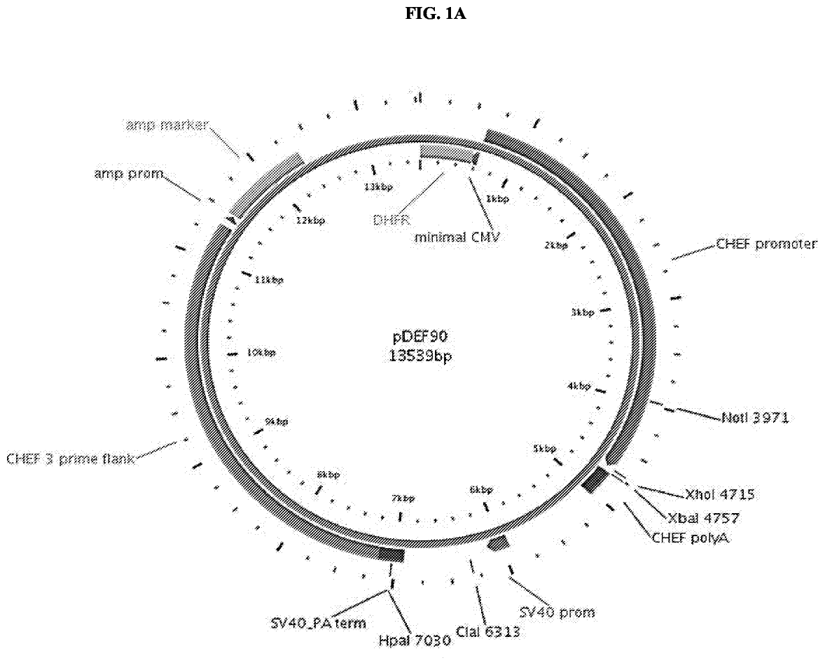

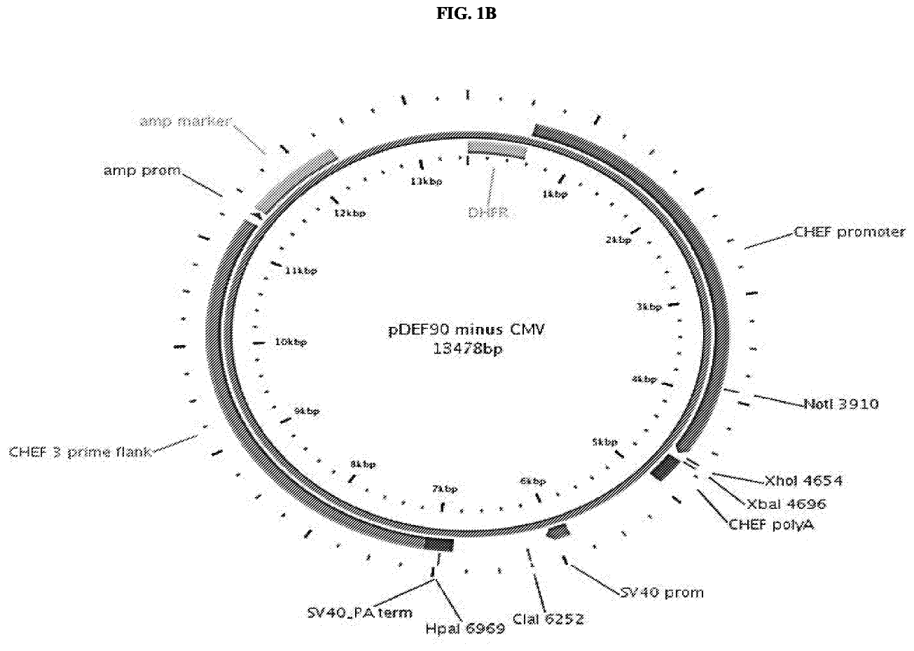

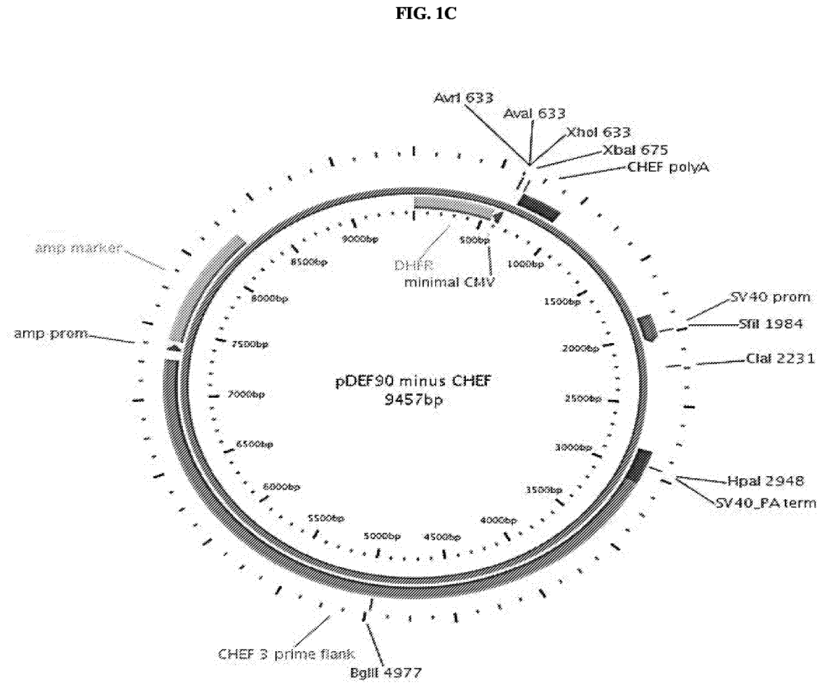

Gene Sequence and Expression Vectors—DNA fragments (61 bp) of the minimal CMV element (SEQ ID NO: 6) was cloned in the minus strand upstream of CHEF promoter (with the DHFR gene cassette downstream of SV40 promoter re-cloned in the minus strand downstream of minCMV element), chemically synthesized and cloned into pDEF38, a CHEF1 expression vector previously described sequence in U.S. Pat. No. 9,297,024 and provided in the instant application as SEQ ID NO: 7, creating a bidirectional CHEF1-minCMV-promoter vector designated pDEF90 (FIG. 1A). Two further variation of the pDEF90 vector were also generated. The bidirectional CHEF1-promoter vector (with the minimal CMV element removed and the DHFR gene present in the reverse orientation upstream of CHEF promoter) designated pDEF90 minus CMV (FIG. 1B) and the pDEF90 minus CHEF (with the 4 kb of 5′ CHEF region removed and the DHFR gene present downstream of minimal CMV element) (FIG. 1C).

All plasmids were prepared using standard molecular biology techniques (Maniatis et al., J. Molecular Cloning: A Laboratory Manual. Cold Spring Harbor Laboratory. 545, 1982). The DNA sequences of the respective plasmids are provided in the sequence listing. pDEF90 (SEQ ID NO:8); pDEF90 minus CMV (SEQ ID NO:9); pDEF90 minus CHEF (SEQ ID NO:10); pDEF93CMV (SEQ ID NO:11); pDEF94CMV (SEQ ID NO:12).

CHO cell culture and media—The cell line used for this project was a serum-free, suspension-adapted CHO cell line designated SFS A. The SFSA cell line was derived from the DG44 cell line, a dhfr mutant CHO cell line. The origin of the DG44 cells has been described by Urlaub, G. et al., Effect of Gamma Rays at the Dihydrofolate Reductase Locus: Deletions and Inversion. Somatic Cell and Molecular Genetics, 12:555-566, 1986).

CD-CIM1 and BM18 media were used throughout cell line development. SFSA cells were passaged in CD-CIM1:BM18 (75:25) blended media with the addition of a 1/100 volume of 200 mM L-glutamine (Gibco, Carlsbad, Calif.) and 1/100 volume of HT Supplement (Gibco, Carlsbad, Calif.) prior to transfection. Subsequently, transfected pools or clones were passaged entirely in CD-CIM1 selective media lacking hypoxanthine and thymidine.

Unless noted otherwise, the SFSA cell cultures were passaged on a 3-day schedule at 37° C. in 5% CO₂ shaking at 125 rpm. Growth cultures were typically seeded at 0.3 or 0.4×10⁶ c/ml. If the culture split was greater than 1:3 then the passage was done by dilution, but if the split was less than 1:3 the passage was done by centrifugation followed by suspension of cells in fresh media. One day prior to electroporation, cells were seeded at 1×10⁶ c/ml.

### Example 1: Transfection of Cells with Various Vector Backbone Plasmids

SFSA DG44 cells were transfected with various plasmids (pDEF38, pDEF90, pDEF90 minus CMV and pDEF90 minus CHEF) using A Biorad electroporator. Briefly, logarithmically growing cells were re-suspended in a DNA/HEBS buffer mix and transferred to a 4 cm gap cuvette. Twenty million cells were transfected with 100 μg of plasmid. Cells were electroporated using standard CHO settings. After electroporation, cells were allowed to recover for 3 days in non-selective media before being transferred to selective media lacking Hypoxanthine and Thymidine by complete medium exchange. Cells were passaged regularly (between 2-4 days) in the selective media. Transfected cells were considered to have fully “recovered” from selection when the culture viability reached greater than 90%.

After transfection, culture viabilities dropped and cell growth slowed as expected during the initial passages in selection media. Approximately two weeks post selection, culture cell growth, viability, doubling time and generations of cells transfected with pDEF38 (pDEF38-1), pDEF90 (pDEF90-1) and pDEF90 minus CMV (pDEF90 minus CMV-1) recovered to >90% as shown in FIGS. 2A-2D, Cells transfected with pDEF90 minus CHEF (pDEF90 minus CHEF-1) and mock transfected cells did not recover in selective media.

These results demonstrated that the pDEF90 plasmid was able to produce sufficient DHFR for cells to grow in selective media, when compared to Mock control with the pDEF90 plasmid. Recovery time for cells in selective media was comparable to the pDEF38 plasmid. CHEF region of pDEF90 plasmid was required for DHFR expression from pDEF90 backbone as plasmid lacking the CHEF promoter was unable support growth in selective media (pDEF90 minus CHEF). The CHEF promoter alone was able to drive gene expression from DHFR cassette cloned upstream in the reverse orientation to support growth in selective media (pDEF90 minus CMV), though the recovery period was slightly longer than pDEF90 and pDEF38 plasmids.

### Example 2: Transfection of Cells with pDEF90 GFP Plasmid

SFSA DG44 cells were transfected with the pDEF90-GFP plasmid (duplicate transfections). Prior to electroporation, pDEF90 plasmid was linearized using Pvul enzyme. DNA was purified by ethanol precipitation and resuspended in autoclaved water for injection (WFI). Logarithmically growing cells were resuspended in DNA/HEBS buffer mix and transferred to a 4 cm gap cuvette. Twenty million cells were transfected with 100 μg of plasmid DNA. Cells were subsequently electroporated using standard CHO settings. After electroporation, cells were allowed to recover for 3 days in non-selective media before being transferred to selective media lacking Hypoxanthine and Thymidine by complete medium exchange. Cells were passaged regularly (between 2-4 days) in the selective media. Transfected cells were considered to have fully “recovered” from selection when the culture viability reached greater than 90% (FIGS. 3A and 3B). Cells from recovered pools were used to analyze for GFP expression using Guava flowcytometer and associated Express Pro software. Cells transfected with empty vector pDEF90 served as negative control for GFP expression (FIG. 3C).

These results how that pDEF90 plasmid backbone is able to sustain CHO cell growth in selective media and drive expression of a heterologous protein.

### Example 3: Cell Line Cloning and Clone Expansion

Cells from one pDEF90-GFP transfection pool were cloned by limiting dilution cloning method. Cells from the pool were resuspended in cloning media by vigorous pipetting to dissociate any multi-cell clumps, and seeded at a theoretical 0.5 cell/well into 96-well plates. A total of 5 plates were seeded. Cloning media consisted of a blend of the following components: 6.2% BM18, 34.4% DMEM/F12, 34.4% CD-CIM1 and 25% conditioned medium isolated from the pools after 3 days of growth. To collect conditioned media, cells were pelleted by centrifugation and supernatant was filtered through 0.2 micron PES filter. Monoclonal cell lines were confirmed to be derived from a single cell by serial imaging through a 14-day period using a Cell Metric Imager from Solentim. Wells containing a single colony were identified and monoclonality of each clone was proved or disproved by identification of a single cell at the colony point of origin in the day 0 image.

Colonies from the 96-well plates exhibited morphologies and growth rates consistent with past antibody and recombinant protein projects run through the CMC Biologics cloning platform. Twelve selected monoclonal colonies were expanded into 6 well plates. These plates were incubated shaking at 125 rpm at 37° C. in 5% CO₂. Once sufficient cell densities were achieved, each clone was analyzed for GFP expression using Guava flowcytometer and associated Express Pro software. Untransfected cells served as negative control for GFP expression.

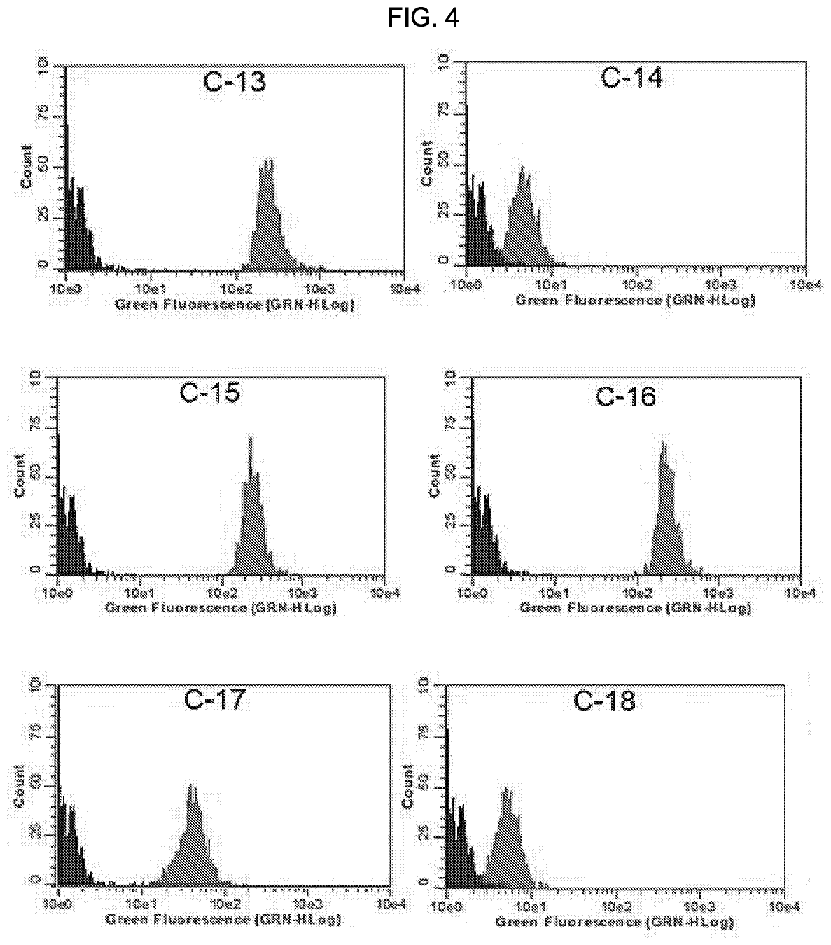

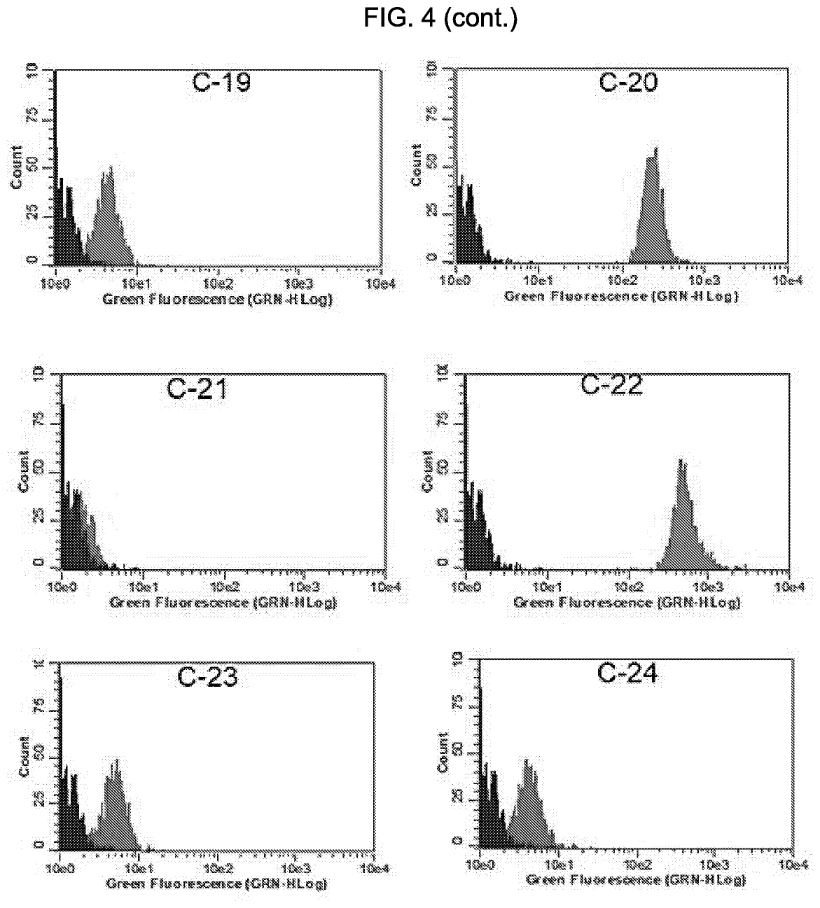

Eleven out of twelve clones scaled into 6 well plates, were positive for GFP expression with varying levels of fluorescence (FIG. 4).

All of the compositions disclosed and claimed herein can be made and executed without undue experimentation in light of the present disclosure. While the compositions of this disclosure have been described in terms of specific embodiments, it will be apparent to those of skill in the art that variations of the compositions can be made without departing from the concept and scope of the disclosure. More specifically, it will be apparent that certain polynucleotides which are both chemically and biologically related may be substituted for the polynucleotides described herein with the same or similar results achieved. All such similar substitutes and modifications apparent to those skilled in the art are deemed to be within the scope and concept of the invention as defined by the appended claims.

The references cited herein throughout, to the extent that they provide exemplary procedural or other details supplementary to those set forth herein, are all specifically incorporated herein by reference.

What is claimed:

1. A bidirectional expression vector comprising Chinese Hamster Elongation Factor-1a (CHEF1) transcriptional regulatory DNA, a gene of interest (GOI), a minimal cytomegalovirus promoter (minCMV) and a selectable marker (SM).

2. The bidirectional expression vector of claim 1, wherein the orientation of the CHEF1 transcriptional regulatory DNA and the GOI are 5′: 3′.

3. The bidirectional expression vector of claim 1, wherein the 5′ CHEF1 transcriptional regulatory DNA comprises Sequence ID NO: 3 or a polynucleotide at least 95% identical to Sequence ID NO: 3.

4. The bidirectional expression vector of claim 1, wherein the 5′ CHEF1 transcriptional regulatory DNA comprises Sequence ID NO: 4 or a polynucleotide at least 95% identical to Sequence ID NO: 4.

5. The bidirectional expression vector of any one of the preceding claims, further comprising 3′ CHEF1 transcriptional regulatory DNA wherein the 3′ CHEF1 transcriptional regulatory DNA is in the same orientation as the 5′ CHEF1 transcriptional regulatory DNA and the GOI.

6. The bidirectional expression vector of claim 5, wherein the 3′ CHEF1 transcriptional regulatory DNA comprises Sequence ID NO: 5 or a polynucleotide at least 95% identical to Sequence ID NO: 5.

7. The bidirectional expression vector of claim 1, wherein the orientation of the minCMV and the SM are 3′: 5′.

8. The bidirectional expression vector of claim 1, wherein the SM is codon deoptimized.

9. A bidirectional expression vector comprising a CHEF1 transcriptional regulatory DNA, a GOI, and a SM.

10. The bidirectional expression vector of claim 9, wherein the orientation of the CHEF1 transcriptional regulatory DNA and the GOI are 5′: 3′.

11. The bidirectional expression vector of claim 9, wherein the 5′ CHEF1 transcriptional regulatory DNA comprises Sequence ID NO: 3 or a polynucleotide at least 95% identical to Sequence ID NO: 3.

12. The bidirectional expression vector of any one of the preceding claims, further comprising 3′ CHEF1 transcriptional regulatory DNA.

13. The bidirectional expression vector of claim 12, wherein the 3′ CHEF1 transcriptional regulatory DNA comprises Sequence ID NO: 5 or a polynucleotide at least 95% identical to Sequence ID NO: 5.

14. The bidirectional expression vector of claim 9, wherein the orientation of the SM is 3′: 5′.

15. The bidirectional expression vector of claim 9, wherein the SM is upstream of the CHEF1 transcriptional regulatory DNA.

16. The bidirectional expression vector of claim 9, wherein the SM is codon deoptimized.

17. A bidirectional expression vector comprising CHEF1 transcriptional regulatory DNA and a CMV promoter and/or a human adenovirus tripartite leader (AdTPL) sequence, a GOI, a minCMV and a SM.

18. A bidirectional expression vector comprising CHEF1 transcriptional regulatory DNA and a CMV promoter, a GOI and a SM.

19. The bidirectional expression vector of claim 17, wherein the SM is codon deoptimized.

20. The bidirectional expression vector of claim 18, wherein the SM is codon deoptimized.

21. The bidirectional expression vector of any of the preceding claims, wherein the SM is selected from the group consisting of neomycin phosphotransferase (npt II), hygromycin phosphotransferase (hpt), dihydrofoate reductase (dhfr), zeocin, phleomycin, bleomycin resistance gene ble (enzyme not known), gentamycin acetyltransferase, streptomycin phosphotransferase, mutant form of acetolactate synthase (als), bromoxynil nitrilase, phosphinothricin acetyl transferase (bar), enolpyruvylshikimate-3-phosphate (EPSP) synthase (aro A), muscle specific tyrosine kinase receptor molecule (MuSK-R), copper-zinc superoxide dismutase (sod1), metallothioneins (cup1, MT1), beta-lactamase (BLA), puromycin N-acetyl-transferase (pac), blasticidin acetyl transferase (bls), blasticidin deaminase (bsr), histidinol dehydrogenase (HDH), N-succinyl-5-aminoimidazole-4-carboxamide ribotide (SAICAR) synthetase (ade1), argininosuccinate lyase (arg4), beta-isopropylmalate dehydrogenase (leu2), invertase (suc2) and orotidine-5′-phosphate (OMP) decarboxylase (ura3).

22. A method for increasing heterologous protein expression in a host cell comprising the steps of culturing the host cell comprising the bidirectional expression vector of any of the preceding claims.

23. The method of claim 22, wherein the host cell is a eukaryotic cell.

24. The method of claim 22, wherein the host cell is a prokaryotic cell.

25. The method of claim 24, wherein the host cell is Escherichia coli.

26. The method of claim 22, wherein the host cell is a yeast cell.

27. The method of claim 26, wherein the host cell is Saccharomyces cerevisiae.

28. The method of claim 26, wherein the host cell is Pichia pastoris.

29. The method of claim 22, wherein the host cell is an insect cell.

30. The method of claim 29, wherein the host cell is Spodoptera frugiperda.

31. The method of claim 22, wherein the host cell is a plant cell.

32. The method of claim 22, wherein the host cell is a protozoan cell.

33. The method of claim 23, wherein the host cell is a mammalian cell.

34. The method of claim 23, wherein the host cell is a human cell.

35. The method of claim 23, wherein said host cell is of Chinese hamster cell.

36. The method of claim 23, wherein said host cell is a Chinese hamster ovary cell (CHO).

37. The method of claim 23, wherein said host cell is a serum-free, suspension-adapted CHO cell line (SFSA DG44).


SEQUENCE LISTING (12 sequences)

SEQ ID NO: 1 - DNA, 676 bp, Homo sapiens
  misc_feature: CMV
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggac

SEQ ID NO: 2 - DNA, 173 bp, Artificial Sequence
  Synthetic polynucleotide
  misc_feature: AdTPL
actctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcga

SEQ ID NO: 3 - DNA, 11716 bp, Cricetulus griseus
  misc_feature: 5′ CHEF 1
ctagaagatatatttgaaatctttactattttgttttctgaaatcacaggacaggggctttgctcttcagcctcgtgggctctgatacttttcctcacatataaagctagttatttttggtggatgccattgagcatttattattctagtccagacacagtcagcaaaagctgctatatgttgactgaaattaagtcaattaaggaaatgaggcaataatgtatgacagtacaggtaaaggatctgcagtggctcacaggtgacagacacacaaattgacgtggggggagaagcacctttttttggggggggctggttagtttggagttttgtttattccttccttccctccttcctttcttcctcccttcccttcccttcccttccttccttccttccttccttccttccttccttccttccttccttcctttctttctttctttctttctttctttctttctttctttctttctgtttttgtgtattgctgtgttttagacaggatctcactatgtagtcctggctggcctggaacttactatgtagaccatgctggcctcagattcacagacacctgtctactcttgcttttttgaggaatgagattgaatgtatgcaccatgaccgatcaggttttgctgttgttttagccaggcttgcttaaaatgcaatgtaaaccttcagcattaggctcctgggttctaggattatagtaggtgtaagccatcatgcccagcttgtcactggtcttaaagatatttatttattttatgtgtataattttttgcctgtgtgtatgtctgtgcacaccatgcatgcctggtgaccgtggagctaaaagagacactcggaattggagttgtattagattgtgagccgctctgtgggtgctggaaattaaactgtgtcctcaagaaaagcagctagtcctcttaactgcttagccatctctctagccctgtccatattttcaaatagaataattgaactgaaagtacctctctgactattggtttcaggattgtacagaataatttgtctgcaatgtcattttaataaattctgcatacttattggaaaacacttttttttaaaactaaactaaaattatctttgatacttttctctctcatcaatatccacttcccttcatttttctatattttcagtaccggggattaaataccaaggttcttttaaatactagattttagctatgactgtacctcatacaaaacctcttgtcagctgtcaagaggtttgatgatcccctggttcttgttgttgtttgaggttgttgtttgagccctgccctggctttcctagaactcacagtgtagaccagtatgaacttgaactcacagtgagcgtccacccacctgcctctgccttctgagtgctgggactaaaggtgtgaaccaacattgcaggcaggaggattcttgattctttgagagtctgttattagaaaaagatgctctaagaatttaactataataacacccagctgagacctggttttctctcatgacatatgcaaagtttgggtgttttttttcaaccaactctggaaagttcctttggcagcaggaatagcttgctaattgatataaggaagcagatggtactagaaaaacagcaacaactaaaaaagataccatacttcgaaagaaaaaagataaatgacatttaaccacagagcacaagtgtgtgtgacaagattttagaattaagggtgactgtttatgttgcttttaaaagacatgtaatttcgggatggtggtggcacacgcctttaatcccagcactcgggaggcagaggcaggtggatctctgtgagttcgagaccagcttggtctacaagagcgagttccaggacagcctccaaagccatagagaaaccctgtctcaacatcccccccccaaaaaaaagaattaggggaactgattatgttgctttttaaaagacatgtaactttcgtagtggtggtggcacatgcctttaatcccagcacttgggtggcaaaggcaggcagatctctgagtgtttaaggacaacctgggttatatagtgagaatctgttctgtcttttaaaaaggtgatagttcactgtacaacctctgacaattatttagtatgttttcatattaccagttacttttttttgtttgtttggtttttcgagacagagacagggtttctctgtgtagctttggagcctatcctggcactcgctctggagaccagggtggcctcgaactcacagagatccgcctgcctctgccttccgagttctgggattaaaggcgtacgccaccaacgcccggctaccagttacttttaaatgtttataattgccaggtggtgatcgtgcatgcttttaatcccagaacttggaaggcagaggcaggcctgtgagtttgaggccagcctggtataaagggctagttccaggacagctagggctattacacagagaaactctgtttcaaacaaacaaacagaaaaacaacaacaatttataattaaaaagatacgaatgagtttttctggactgactcaaaataatgtcataaaacatacaaattgagagtagtctttcctcagaagtcctataggcacatgtcttcctagctaacaattgtacaatgtcactttatgtcatggcatattggaactgtgaggtagacgctaagaataaggagccatctgctttgtagattcgcttgagagatctacaactgccagttgtcttgaagtctgaacaaaggcaagtgtagccttggcccctaattatcaggctccagaaatactgctagtttcattatgccagaccccatctgaattctaattctttgctagaccccgatagagtcccctccccccaaaaaaaagattgacaagatgcagtaaatgaaaacagtacggtgatatgctgtattttattattttgtgtaaaaatagcaatacttcacaaaagaatgcatatgcttatgtataaactattgtggcgtatccactgatccaggcaaaggcctgaagaaaagatagacacagacagaaagctggggattggctggtctgggctccctgatggagaagctgcagcaaccagaaagctcgtgtgtttattctaaagagctgaatagggaggcgggttactcagtacagagaaacaagaagacagagtttacagtatacagctggaccaaggagacaggtgtagctaatctgagtaggaactctctctctgtaggggagaattctctaggctataaatatgagggtggagtcaaaagctttggtggacatttttttttttttttaactctgtcaacacttggactcagtattcctccaggaaagatttgctatctttgcttacatggccctgcctatgtcatcaataaacattcgctcagggcttcctctgctcccaacgctaatgcttgcaacttctgcatctctaaaatgttggtagtgagactgcattatagactgcaaattttaagagacggccttccacacaggtgggccgatttttagttggattatcagttcaaaacattatttaattgcctcctcccctctctgtctctgactctgtctctctttcggtggtttgttttgtttttatatagggttggactatgtagtttctatatagaacaagttggccttgaactcacagagagccacctgcctctgcctccaaatgctgggattaaagtcctgtgccaccacttcctggcctaatcctactcttttatttatttattttatgtgtatatatgtctgcttatatgtctgtgtgaaagtgtcagatcccctggaactggtgttacagacagttgtgagctgcaatgtgggtgctggggattgaacctgaatcttctggaagagcagccagtgcccttaactgttgagccatccctccagtcctcctaatcctactctgaaatacccaaagtcactttggtcttgcactcctgtaattccagcacctaggggctggaggccagaagttttggtgtttaaggccaattttggttacttagggagttcaaggtcagctccagttacatgagaagagggaagaaagcagggagaaggagagaggttaagaatatgaaaatgtgagaatctggaagtaaggccttgtgtagccagaacagctctttacagctgagctgcatatccagctctataaaatccttttcaaaatctgagtaagtacggtgagtctgagtgagcacggtgcagcaggactatattcccagtatttaagaagttgtttggaagagcccctgttcaaaaccagcctgactatgtagtgaggaaatcattttctttttgttgttgttgttctgttttgtttgagacagggtttcttggtgtagtcttggctgtcctggactctctgtagacagggctggtcttcaactcatagagataacacctacaatgcctcctgagggctggtgtgtgtgtgtgtgtgtgtgtgtgtgtgtgtgtgtgtgtgtgtgtgtgtgtgtgtgtgtagatatagtctcaacaaagtcagtttttagcactgattttagtaaaaagcatacggaatctgctgtaacatgaagcaggggaggaggagccaaaatgaagtaaggttgcactctaaatgccggttgcacaaaacatgcttttaatgaatccctatataaccagtcttttcgaagcaaagcacttgaacctttccatcctccaggtgtcttctgcagactgaggacacacgaaactgaccttaggtggggggcgggtggaggggtggcctgggccacctcattcttgggagaacagaaatggctctcggtgaggcaggtggtaagcaggtgcactggcgacaaggagacgcttcgaccccacccgcgggggaaagggaggggatgggaaaggtggggaggggatgggaggggtggggaggggaggggaggggaggggaggggaggggaggggaggggactgcggggcctcccaaccccccgaagcggaagtggctccctgccaagagctcaggctgtggccagtgtctaccagaaatacgtaaacattgagtctcctgggctaaaaggtatctacacttaaacacgagactcacgatttcactttctggtatagaaaatcccagccattatctaacttaaggatcaccttaagttaggttacttagaaactaaggagtcttgtgtgatttttctttttctttctgatatcttagatagcatcctccatatggcgtttataaagtgtatgctctcattctttaatatctaagaaaagacctacattctaggatagcttcgggaggagtgattcaacttttggatatttcttttctgcctttctttcttagcataaccataatatgagctgtacctggtgacacaggcgtgtcagctgcccgggatgctgcccgggatgctgaggcaagaccatctcaagctgggcaatttagcaagaccctgtctcaaaacacaaagcaaagcagggtgcgaaacccacagttgaaatcgtagcacttagtaagaaaagggctaggctatgtgaaactgcctcaaaattcaagggactaaaatattttttaactgcactagaatacagatacaatgctggcttagcatgtgcaaggacctaggttcagtctccactatcaaacaacaacagtaataacagtaacagtggcgaaaataaacggctgaatctcatttttgtttcaagagcaattttgtatcaatttgtgacttaattatatttagaatgttggattttttttttaaccataggcaaaagcttaagacatatttcaatcaggaaaagtacttttcatcagctgtggaaatatagtttcttaaagaaatgcaacactggacatacacacaaacacacctctataagacagtttgcattaggtccatatattttaaaaattagattcactcattttattttaaaagtgtttccttacaggtatgtatgtgcaccatgcccatactttgtactcaggaaggtcaggagaaggcacagttccccaggaactgaagttacagaaggttggagggttttgagccatcacatgggttctgggaattgaacctgggtcctcggcaaaagctgctcttaactgctgagccatatctccagcccttatttttacataggctctctctatgtagtcctgactgttttagaacttgatatgtaggccaggctagcctcgaactcatagagatccttctgcctctgcttcctgagtgggttacctttggaggccagagacatcaggttccccagaaaatgggaactgaacttgtatgttcagcaagaccggttaaataaatgctcttaagggctggttcctctctccagcccttatttttattcttttttaattatatgtaggtgtgttatatgtctgcactgtagatttgtgccaaggaatgcaggtgccttgggaggcccaaggtgttggatccccaagaactgggagctgatcttctgccctctggaagaacagcaagtgctcttcaccactgagccatttctccagccctgtccttttattgttctttttgtttttttgagacaggctttctctgtgtagccttggctgtcctggatctttctctgtagaccaggctggcctcaaattgaactgaaagcatatctctaatcattagtttcaggattgtacagaatcattctgcatacttcttggaaattaccttttttttttttttaactagaatcatctttgatgcttttctttttcactaatatctactgtaatttttccccatttctttcagtactggggattaaatatcaagactctctttaaggattagattttagccttgattctggacctcatataaaactttcaaaagctcaaccttcagtgttcaggactgaagaagactagtcgcagttgctagagtgcttgcctagcatttgtgaagcgtgggttgggttcctcagcactgtattaacaccatgcatggagatgaatgcctgtatcctcagcacttgagaggtagaggcgggggaatcagaaatccaaggttatcctcagttacataatgagtttgaggtcagcctaggctatatgaggctctgtctcaagaaaacaaactagccaggtggttgtttgtttgattaaaggcacacgcctttaatcccagcacttgggaggcagagacaggccgatctctgtgagttcaaggccagtctggtctccagagtaagtgccaggataggctccaaagctacacagagaaaccatgtctcgaaaaaccaaaaaaagaaagaaagaaaacaaacaaacaaatagaacagaacaaaggccagacccccaagcttgggaggcagagtcaagaggatttctgaggccagcatagtaagttctaggccagccagggctgcatagtaaaatccctcagaaaacaaacaagtaacacacaacaaatagggctggagagatggatcaacagttaagagcacttggttctcttgcagaggacagttcagttcccagcgcccagatcaggcagcacacaacattctgtagtagctagctccaggagacctgacaccctgacctctgaggacaccaggcacacacgttgtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcaggggatggcggggctgacgtcgggaggtggcctccacgggaagggacacccggatctcgacacagccttggcagtggagtcaggaagggtaggacagattctggacgccctcttggccagtcctcaccgccccacccccgatggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcggaccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgagcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaag

SEQ ID NO: 4 - DNA, 4057 bp, Cricetulus griseus
  misc_feature: 5′ CHEF 2
gtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcagggggatggcggggctgacgtcgggaggtggcctccacgggaagggacacccggatctcgacacagccttggcagtggagtcaggaagggtagggacagattctggacgccctcttggccagctcctcaccgccccacccccgactggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcgggccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgaggcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaag

SEQ ID NO: 5 - DNA, 4180 bp, Homo sapiens
  misc_feature: 3′ CHEF
tccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactaccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaatgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaacgtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagggaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctaggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagt

SEQ ID NO: 6 - DNA, 61 bp, Artificial Sequence
  Synthetic polynucleotide
  misc_feature: Minimal CMV
aggtaggcgtgtacggtgggaggtctatataagcagagctggtttagtgaaccgtcagatc

SEQ ID NO: 7 - DNA, 13478 bp, Artificial Sequence
  Synthetic polynucleotide
  misc_feature: pDEF38
tcgacggcgcggtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcagggggatggcggggctgacgtcgggaggtggcctccacgggaagggacacccggatctcgacacagccttggcagtggagtcaggaagggtagggacagattctggacgccctcttggccagctcctcaccgccccacccccgactggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcgggccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgaggcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaagcttctcgagcgtacgtattaattaactcacgcgtattggcgcgcctctagaatattacccctaacacctgccaccccagtcttaatcagtggtggaagaacggtctcagaactgtttgtctcaattggccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagagtcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtgggtacctctagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggggggggacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatcgatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccggccgcgtttccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaagtgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaaggtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagtgaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctagggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctc

SEQ ID NO: 8 - DNA, 13539 bp, Artificial Sequence
  Synthetic polynucleotide
  misc_feature: pDEF90
ttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatctgacggttcactaaaccagctctgcttatatagacctcccaccgtacacgcctaccttcgacggcgcggtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcagggggatggcggggctgacgtcgggaggtggcctccacgggaagggacacccggatctcgacacagccttggcagtggagtcaggaagggtagggacagattctggacgccctcttggccagctcctcaccgccccacccccgactggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcgggccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgaggcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaagcttctcgagcgtacgtattaattaactcacgcgtattggcgcgcctctagaatattacccctaacacctgccaccccagtcttaatcagtggtggaagaacggtctcagaactgtttgtctcaattggccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagagtcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtgggtacctctagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggggggggacagctcagggctgcgatttcgcgccaaacttgacggcaatcctagcgtgaaggctggtaggattttatccccgctgccatccaggaagatgctttcaagttctctgctcccctcctaaagctatgcatttttataagaccatgggacttttgctggctttagatcgatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccggccgcgtttccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaagtgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaaggtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagtgaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctagggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctc

SEQ ID NO: 9 - DNA, 13478 bp, Artificial Sequence
  Synthetic Polynucleotide
  misc_feature: pDEF90 minus CMV
ttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccattcgacggcgcggtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcagggggatggcggggctgacgtcgggaggtggcctccacgggaagggacacccggatctcgacacagccttggcagtggagtcaggaagggtagggacagattctggacgccctcttggccagctcctcaccgccccacccccgactggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcgggccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgaggcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaagcttctcgagcgtacgtattaattaactcacgcgtattggcgcgcctctagaatattacccctaacacctgccaccccagtcttaatcagtggtggaagaacggtctcagaactgtttgtctcaattggccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagagtcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtgggtacctctagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggggggggacagctcagggctgcgatttcgcgccaaacttgacggcaatcctagcgtgaaggctggtaggattttatccccgctgccatccaggaagatgctttcaagttctctgctcccctcctaaagctatgcatttttataagaccatgggacttttgctggctttagatcgatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccggccgcgtttccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaagtgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaaggtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagtgaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctagggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctc

SEQ ID NO: 10 - DNA, 9457 bp, Artificial Sequence
  Synthetic Polynucleotide
  misc_feature: pDEF90 minus CHEF
ttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatctgacggttcactaaaccagctctgcttatatagacctcccaccgtacacgcctacctaagcttctcgagcgtacgtattaattaactcacgcgtattggcgcgcctctagaatattacccctaacacctgccaccccagtcttaatcagtggtggaagaacggtctcagaactgtttgtctcaattggccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagagtcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtgggtacctctagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctagcttggggggggggacagctcagggctgcgatttcgcgccaaacttgacggcaatcctagcgtgaaggctggtaggattttatccccgctgccatccaggaagatgctttcaagttctctgctcccctcctaaagctatgcatttttataagaccatgggacttttgctggctttagatcgatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccggccgcgtttccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaagtgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaaggtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagtgaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctagggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctc

SEQ ID NO: 11 - DNA, 13338 bp, Artificial Sequence
  Synthetic Polynucleotide
  misc_feature: pDEF93CMV
cgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatcgatctaaagccagcaaaagtcccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatggcagcggggataaaatcctaccagccttcacgctaggattgccgtcaagtttggcgcgaaatcgcagccctgagctgtccccccccccaagctgatctgacggttcactaaaccagctctgcttatatagacctcccaccgtacacgcctaccttcgacggcgcggtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcagggggatggcggggctgacgtcgggaggtggcctccacgggaagggacacccggatctcgacacagccttggcagtggagtcaggaagggtagggacagattctggacgccctcttggccagctcctcaccgccccacccccgactggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcgggccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgaggcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaagcttctcgagcgtacgtattaattaactcacgcgtattggcgcgcctctagaatattacccctaacacctgccaccccagtcttaatcagtggtggaagaacggtctcagaactgtttgtctcaattggccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagagtcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtgggtacctctagcatcggccggccgcgtttccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaagtgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaaggtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagtgaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctagggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctc

SEQ ID NO: 12 - DNA, 13241 bp, Artificial Sequence
  Synthetic Polynucleotide
  misc_feature: pDEF94CMV
cgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatcgatctaaagccagcaaaagtcccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtaggtctccgttcttgccaatccccatattttgggacacggcgacgatgcagttcaatggtcgaaccatgatctgacggttcactaaaccagctctgcttatatagacctcccaccgtacacgcctaccttcgacggcgcggtgcatagaaacagatgcaggcaaaacatccacacatataaaacaaaaaattaaaaccaataaaactcctaaacttttggtctttcttgaatcttcaatccctcaggttatgaaataatcatttatgcagtcaaaaatttgccattcttgttgccaggtgtggtgatgattcggggaagcagaagcaggcagatctctgtgaatgaggccagcctggtctacaaagtgagtcccaggacagtcaggcctgttacacagagaaaccttgaaaaaaaaaagataatatgtactgttgtattaccccaatatataaggctaaaccattagaagcacaacactgttaagtacggaaaataatatctagtgtggtacagttactactactataatacactaatatagctgtgggaaactagttccaaagatgaattactaaccagtgtttccaaggaaataaatgaaagcagagagattagttctattgctagtgtttcattttcgtatatttcttacaatttctcttgttacaaataggcactagggtatcaagataattttaacgactggctgagaaccctagaaaatctctgtgaaaaagggatttgtgaaatgagagagggtaatgtggccattatagaaaaggcttttgtgtgccttgcatgcatagaccctgtgtttgatctcttaacaccctccttgaccagaaaaagcttctgtggatagaaaatgattagttatatatacttttagggaaacgtagttctggattctttggttacaattaacagaattaagtgcaaacaaagccagaaacctcctgataaatgagaaaacctgcttgtagaaggttgtaaggctctgtaatataggaattaggagaaaagaaacctgtgtggtggggcacgtctgtaatcccagcattgggaagtagaggtagaagattagaaatcaaaggccagcctcagcaacacagtgagtttgaggccaccctgaactacatcaggttctgtctcctttcttttttttttttttttcttttctttttttggtttctctgtgtagttttggagcctatcctggcactagctctgaagagcaggctggcctcgaactcagagatcagccagcctctgctgggattaaaggtatgcaccaccaacgccccaggttttgtctcaaacaaacaaaaataacatcaggaggtggtgagagggctcagtggtcacaggcattctctgcaaagcctgactctgagttggatcctttagagctacatggttgagggaagagaactgactcctggaaggtgtcctctggtccccacacatagctatacacagcatgtgcattcacacacactaaataatgctatttttaaaaaaattaaaaacaacaacagtttgggttgtgaaaactagaactagataataggtaagaatcaagtatcatgtaaatttgctttcaactcatcccaaaatttgttttatatttcagtttttttccttcctagcttgactgtggagtcttgtccggaagcaaatagttcctttgcagatcccacatgtggacaccggacagtaggtcctcaaatgctccttattaggttggttcaataatatcaattgtttgttactaggcagtgatgttgtacatctggaggagatctcttgagcccataatcaggttattaggaataaatactctaaggctaaaaatgtagcttagtgataagagtgcttgcctggtgtgctgagaccctcggttccatctccacaaccccatattccattacaaaataccttttcaccgtccctagcattaagaaacaaaacaacaaagaagtttttctttcttctgagatcctgcccggagaggcatttaaaactggccagggccaaaaaaaaaaaaaaaaaaagaaaaaaaagaaaagaaaacaggctagggccggcatggtggcgcacgcctttaatcccagcacgcaggaggcagaggcagggcggatctctgtgagtttgaggtcagcctggtctacctagtgagtttcagggcacccagggctaaagagactgtctcaaaaacaaaacagccacacaatcagaaccacagcaaaacgcagttatgatccttggaactgtaggaatgacaagcatttaaataataggacgagccatttttgagaagctctgatttcacaagtgtcagggatgggctctgggcgagtaagattgctaatgctggcctctaaatgagaccacgtggagttgattagattcttttcatgttcctcgtgctctatcaaataactgtacccaaatacacacacacacacacacacacacaatgcgcgcacacacaaaatccttttttagcttaagaagcccagaatcagaagtaaagctaactgtgggacttaagtattattctgaacggaactcccagggcgtgaagcgcgcttcaggcttccagagaagcagctggcgctggatggaatgaaccaagaggccagcacaggggcagatccgtcgagctctcggccaccgagctgagcccttaggttctggggctgggaagggtccctaggattgtgcacctctcccgcgggggacaagcagggggatggcggggctgacgtcgggaggtggcctccacgggaagggacacccggatctcgacacagccttggcagtggagtcaggaagggtagggacagattctggacgccctcttggccagctcctcaccgccccacccccgactggagccgagagtaattcatacaaaaggagggatcgccttcgcccctgggaatcccagggaccgtcgctaaattctggccggcctcccagcccggaaccgctgtgcccgcccagcgcggcgggaggagcctgcgcctagggcggatcgcgggtcggcgggagagcacaagcccacagtccccggcggtgggggaggggcgcgctgagcgggggcccgggagccagcgcggggcaaactgggaaagtggtgtcgtgtgctggctccgccctcttcccgagggtgggggagaacggtataaaagtgcggtagtcgcgttggacgttctttttcgcaacgggtttgccgtcagaacgcaggtgagtggcgggtgtggcctccgcgggcccgggctccctcctttgagcggggtcgggccgccgtgcgggtgtcgtcggccgggcttctctgcgagcgttcccgccctggatggcgggctgtgcgggagggcgagggggggaggcctggcggcggccccggagcctcgcctcgtgtcgggcgtgaggcctagcgtggcttccgccccgccgcgtgccaccgcggccgcgctttgctgtctgcccggctgccctcgattgcctgcccgcggcccgggccaacaaagggagggcgtggagctggctggtagggagccccgtagtccgcatgtcgggcagggagagcggcagcagtcgggggggggaccgggcccgcccgtcccgcagcacatgtccgacgccgcctggacgggtagcggcctgtgtcctgataaggcggccgggcggtgggttttagatgccgggttcaggtggccccgggtcccggcccggtctggccagtaccccgtagtggcttagctccgaggagggcgaggcccgcccgcccggcaccagttgcgtgcgcggaaagatggccgctcccgggccctgtagcaaggagctcaaaatggaggacgcggcagcccggcggagcggggcgggtgagtcacccacacaaaggaagagggccttgcccctcgccggccgctgcttcctgtgaccccgtggtgtaccggccgcacttcagtcaccccgggcgctctttcggagcaccgctggcctccgctgggggaggggatctgtctaatggcgttggagtttgctcacatttggtgggtggagactgtagccaggccagcctggccatggaagtaattcttggaatttgcccattttgagtttggagcgaagctgattgacaaagctgcttagccgttcaaaggtattcttcgaactttttttttaaggtgttgtgaaaaccaagcttctcgagcgtacgtattaattaactcacgcgtattggcgcgcctctagaatattacccctaacacctgccaccccagtcttaatcagtggtggaagaacggtctcagaactgtttgtctcaattggccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagagtcgaggggggatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtgggtacctctagcatcggccggccgcgtttccatttaagtttaatagtgaaagactggttaatgataacaatgcatcggaaaaccttcaggaggaaaggagaatgttttgtggaacatttttgtgtgtgtggcagttttaagttattagttttcaaaatcagtactttttaatggaaacaacttgaccaaaaatctgtcacagaattttgagacccattaaaatacaagtttaatgagaagtctgtctctgttaatgctgaagtcattactaagtgcttagcttagcaaggtatgtggatgcccatttgtgttccaagggattggactgttcatcaggacccagagctgagtttcaagggctcaagagatggcttattacctgtgggtgtcttgaaggttctggttgggacaaattaggaatgtttttggcagacatggtgactacccttcatctgggtgagttcagttgatttgtcttgagcctttggggtttacacaagtaaatgacatcatacagttagtgtattgttagtgaatattaatatatgaggcaggctttgctctagcaattttagaactagttttcaggaaagggttcatcttgtgcattggatgtttgattctatcacttagagtttaaactgaaagtgctcaagaggttttatttaggctgggatataaataagcctttctgtagcttgtaatggtatcaggaatttaaaaggccatctggggcacaaagattaagcagaaaaggtagaaggtgagattgggggactttgagtacttcacacactttaatgtgtgagtgctttagtgcatatagtacaactgccagataagggcatccacatctgattgtttggaaggcaccttgtggtttctgggaattcagaattgggagaaaaagtgctcccaaccgctgaagcccttggtaatctgcagggtgtttatttagcaggagataaggacaaaaagttatagtgtggagttggttgagttggtagatgtcattacaacaggtggtcttaaattgggttaggagtcactttgaaatacctgggccataagcaaagtggcattttcacctttcaggagaaactggtacacttatccattctatagtgcatgcttgttcaattgggctgatgactaaaccggtgactaaaggtttgtcagtataaatggatgggttgtaggcagacggtgaggaattactatacctgcaaggagtcattgcctgatctgcctggaaaggggcaggattgagtctcagaaggtgtacaccataggatatggaaaaatttgtcacgcctagcattcaacttagtggtgtagcgccacctactggcactttaaaagcttagcatagaggagcatgtgtgttaggagctcggatgggatccagggcctcaaggtttgcatgtaaataaaagccctttaccaaattaactacataccagcatacatcagtcctttagtgttgaaaaacagaagtgaaagctaatatatatagtgcttgctttatttaagtctagctgattacgtgtttggttgccagtgtgactagtctggagttgaatttgtcctcagacacgtaaaatggaatttgggattcacaacactctagtatgagggacctaatggcctgtaccaggcacaaacgtgtctataaactacacaaaacgaaggaatttacaggaattaggaaggtattcttaacattaaaacattatgggcattttaaaaaaagctttgacaggatttctttgtcatggctgtcctggagctagttgtgtagaccaggctgggctgaaatcttgtctgcctgcctggcttggacacttttttattatgtatacaacattctgcttccatgtatatctgcacattagaagacggcaccagatctcctaatggatggttgtgagccaccatgtggttgctgggaattgaactcaggacctctggaagagcagtgctcttaacctctgagccatctccagccccagcttgggcacatttttaatggctgggaaatcaaaccccctaggccttctgtcagtaatgaagggcttttggctaccgagagtaggatttaaggttattcggagctgcaggtctgcctcagtgcaggtttgggagtccagcatcttagaaaatgcagtgaagccaagctgagctatattttgtttaaaaaaaaaataagtgggtaaagtgctgctgagcctgatgaccaagctgggacacaagtagaagaacataggccaatgctctatattaaaagcatgggtcatttttaatgctcttgagaaggctatgcctacactactctcagccaccgcagcgtgtttaaattaaactagtttggaaattttctttgggggtaagctatttaacctagtgccttggcaggtatactactgaactctcctcctcattcctttttgttttttaagaatttcagtcaggctcaggcagcccttaaacttgtgattaagcctgagaacagttacgattatgagcctattagtataccgatcaatatgtgaatttttttgggatgggggtcaggcctccctgcctcccaaatactgggactaaaggctgcaccaccacaacctggctcttgaaatacttttctacattttttggggggcatgggtgggagagcagggtttctctgtattagccctggctctcctggaactctgtagaccaggctattcttgagctcagattagcctgtctctgcctcctaaattctgggattaaaggtgtgtgctactgctgcctggctacaaagacatttttttttttcttaaatttaaaaacaaaagtggttcttttagaagggtggttggtgttggcacatactccaagcactcaggttttgagtttgtcccaggaatgaagactgcattactgccgcccctccctggtaagggctacacagagaaatcctatttggagcctatcctggtaactcgctctgtagaccaggctggcctcgaactcaagagaaccacctgcctctgaatgctggtattaagggcaggcaccaccaacacccagcctaaaaaatgtcttttttttaaagatttttttttttttttttacagaataaacattctgtttacaatattctgcttctatgtatatctgcacactagaagagggcacccgatctcataatggatggttgtgagccaccaagtggttgctgggaattgaactcagaacctctggaagagcagtcagtgctcttaacctctgagccatctctccagcccctaaaaatggctcttgagatagggtctcaagtagtttgagactgagttggctatataaacaaggctggcacatagcaccatgtacagctgggtttagtttacatggggtgtttttgtctctggaggcaggaggatcatttgagcatagggagttaatagtgaggtcatgttttatctactcttctgaattgagaactaagctgatgcaaagcaagtttgactgaagaagtccagtttatgagaacaagggtggaaactaatgtgtcaaagatggccttgcatgtgttttagatgatgacccagtcacttgggaattactggatgtgtaagacctatatcttgacaggagtgaacagtgtcttataggtcctatatgaaagaaatgagacatacccattttgtttcccctaagaattcacttttcctaacctggttcatgctatttaggttattttacttgcaaatcctagggtgctcccttacccagtattgcttatgtggcaccaaagtcactcactcccatgatttgcaagtctctgggaacttccatgacaacctagaatagcaactcaaatacattttctcagtaccaattttgaagaaaaaatattttgcaaaatagctgtatggatgggtactaaatagtgaggttatctccagaaggcctatgaagaattaaggttgagttcagttgagttcagcagcaagtttaaggttcatccatttttgtacagtgttttcctattacggtaagtgttttgcctgcaggaatatctgtaccacatgcttgcctggtacctatatcggccagaagagggctttggatcctctggacttgaattacagatgggtattagccaccatttaggtgctgggaattgaaaccaagtcctctggaagaacagcaagtgatcgagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctc